(19) United States
(12) Patent Application Publication
(10) Pub. No.: US 2024/0390252 A1
(43) Pub. Date: Nov. 28, 2024

(54) COSMETIC COMPOSITIONS AND RELATED METHODS OF USE

(71) Applicant: Younique LLC, Lehi, UT (US)

(72) Inventor: Bihareh Hossein-Beik, Draper, UT (US)

(21) Appl. No.: 18/671,679

(22) Filed: May 22, 2024

Related U.S. Application Data
(60) Provisional application No. 63/503,898, filed on May 23, 2023.

(51) Int. Cl. A61K 8/64; A61K 8/67; A61K 8/81; A61K 8/9789; A61Q 1/10

(52) U.S. Cl. CPC A61K 8/64; A61K 8/673; A61K 8/8111; A61K 8/8152; A61K 8/817; A61K 8/9789; A61Q 1/10; A61K 2800/30

(57) ABSTRACT

The present disclosure relates to cosmetic compositions and related methods of use. The cosmetic compositions can act as a lash serum, primer, and mascara all in one. The cosmetic compositions include a mixture of peptides and vitamins, including myristoyl pentapeptide-1 and biotinoyl tripeptide-1.

### RELATED APPLICATIONS

This application claims priority to U.S. Provisional Patent Application No. 63/503,898, filed May 23, 2023, and titled COSMETIC COMPOSITIONS AND RELATED METHODS OF USE, which is incorporated herein by reference in its entirety.

### TECHNICAL FIELD

The present disclosure is generally directed towards cosmetic compositions. More specifically, the disclosure relates to mascara compositions comprising a mixture of peptides and vitamins that can act as a lash serum, primer, and mascara all in one.

### DETAILED DESCRIPTION

Enhancing the visualization and beauty of one's eyes can be of significant importance to consumers. Enhancing the visibility of eyelashes is often of particular importance, and has led to the development of numerous cosmetic products. For instance, lash serums have been developed to stimulate lash growth and/or hydrate and condition lash hair. Lash primers have been developed to coat and prepare lashes for the application of a mascara. And mascara's have been developed to enhance the visualization and beauty of a person's eyes by thickening, lengthening, darkening, lifting, and/or shaping (e.g., curl) the eyelashes.

Disclosed herein are cosmetic and/or mascara compositions that can act as a lash serum, primer, and mascara all in one. Methods of using the cosmetic compositions are also disclosed.

In some embodiments, the disclosed compositions comprise a mixture of peptides and vitamins that can enhance a person's lashes for a prolonged period of time. For instance, the compositions disclosed herein can aid in extending the duration of the anagen phase of hair growth so that the lashes can grow for a longer period of time. The disclosed compositions also create supple, non-brittle, nourished lashes that will not easily break or fall out during the catagen phase of hair growth, creating a fuller lash line. The compositions are also easily removable, so that little to no additional lashes are lost beyond what is expected in a healthy hair growth process. The compositions can also be prostaglandin free, avoiding the side effects associated with prostaglandin use. Additionally, the compositions can be paraben free, sulfate free, and formulated without phthalates.

In some embodiments, the compositions can comprise tube or tube-like fibers or polymers and can be considered a tubing mascara. Tubing fibers or polymers can wrap around individual lashes to create a tubing effect, which differs from traditional mascaras that paint lashes with a pigment. This tubing effect can provide length, volume (or bulk), and/or lift to the lashes. Tubing mascara's can also keep lashes individually defined and free of clumps. The compositions can also include star-shaped fibers or polymers. The star-shaped fibers or polymers can add length, volume (or bulk) and/or curl to the lashes. The tubing and star-shaped fibers or polymers can be used individually, or in combination.

An exemplary tubing fiber or polymer that can be used includes a tubing fiber or polymer that comprises VP/Eicosene copolymer (2-pyrrolidinone, 1-ethenyl-, polymer with 1-eiocosene). In certain embodiments, the composition comprises from about 3% to about 5% by weight of VP/Eicosene copolymer. An exemplary star-shaped fiber or polymer that can be used includes a star-shaped fiber or polymer that comprises nylon 6 (or polycaprolactam), such as about 0.1% to about 0.5% nylon 6 (polycaprolactam) by weight of the composition. In certain embodiments, the star shaped-fiber comprises a mixture of nylon 6 (or polycaprolactam), silica, and iron oxides. In some of such embodiments, the composition comprises a mixture of star shaped-fiber comprising about 0.1% to about 0.5% nylon 6 (polycaprolactam), about 0.001% to about 0.003% silica, and about 0.001% to about 0.003% iron oxides by weight of the composition.

The mascara compositions disclosed herein are easy to apply and remove. For instance, to remove, one simply rinses off the compositions with warm water and a washcloth (e.g., such as with a gentle downward-sweeping motion). The mascara compositions will rinse off each lash, without leaving mascara streaks behind. If desired, makeup remover can also be employed, although it may not be necessary.

As previously discussed, the compositions disclosed herein contain a mixture of peptides and vitamins, some of which can be considered active ingredients. In some instances, the active ingredients in the composition can include one or more peptides (or peptide mixtures). In certain of such embodiments, the composition comprises two or more peptides (or peptide mixtures). For example, the composition can comprise a pentapeptide and a tripeptide (or mixtures that include a pentapeptide and a tripeptide). In certain embodiments, the composition comprises a mixture of myristoyl pentapeptide-17 and biotinoyl tripeptide-1. And in some of such embodiments, the composition comprises SYMPEPTIDE® Xlash (which comprises myristoyl pentapeptide-17), WIDELASH™ (which comprises biotinoyl tripeptide-1), or a combination thereof.

In some embodiments, the composition comprises from about 0.001% to about 0.01%, or from about 0.005% to about 0.01%, by weight of myristoyl pentapeptide-17. In embodiments comprising SYMPEPTIDE® Xlash, the composition comprises a mixture comprising about 3% to about 5% water, about 0.5% to about 1% glycerin, about 0.005% to about 0.01% myristoyl pentapeptide-17, about 0.005% to about 0.01% benzoic acid, and about 0.005% to about 0.01% sodium benzoate by weight of the composition.

Myristoyl pentapeptide-17 has the following structure:

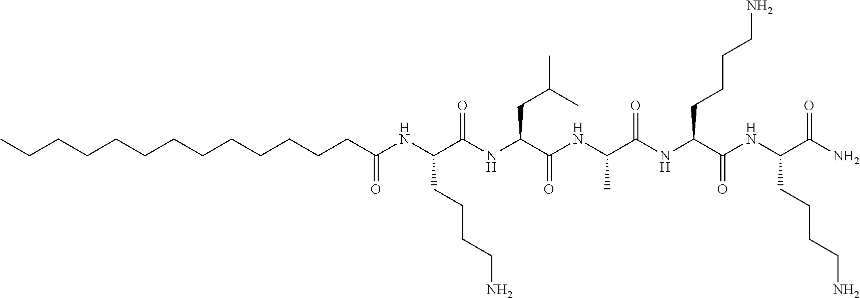 [SMILES: CCCCCCCCCCCCCC(=O)N[C@@H](CCCCN)C(=O)N[C@@H](CC(C)C)C(=O)N[C@@H](C)C(=O)N[C@@H](CCCCN)C(=O)N[C@@H](CCCCN)C(N)=O]

As shown in the structure above, myristoyl pentapeptide-17 comprises a chain of five peptides (lysine-leucine-alanine-lysine-lysine) attached to myristic acid, an oil-soluble fatty acid component. Myristoyl pentapeptide-17 can promote keratin production (a protein that makes up hair) and lengthen the hair growth cycle. Myristoyl pentapeptide-17 can also stimulate hair growth at the follicle and promote lash growth. When used in the compositions disclosed herein, myristoyl pentapeptide-17 can aid in producing longer and/or thicker lashes.

In some embodiments, the composition comprises from about 0.001% and about 0.01%, or from about 0.005% and about 0.01%, by weight of biotinoyl tripeptide-1. In embodiments comprising WIDELASH™, the composition comprises a mixture comprising about 1% to about 3% glycerin, about 0.1% to about 0.5% water, about 0.01% to about 0.03% panthenol, and about 0.005% to about 0.01% biotinoyl tripeptide-1 by weight of the composition.

Biotinoyl tripeptide-1 has the following structure:

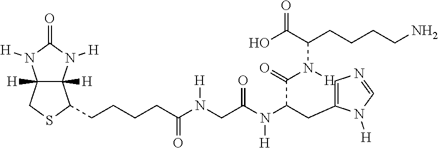

Biotinoyl tripeptide-1 can aid in reducing lash hair loss and in promoting a thicker appearance for one's lashes. Biotinoyl tripeptide-1 also has a positive impact on stimulating lash growth. When used in the compositions disclosed herein, biotinoyl tripeptide-1 can aid lengthening lashes, strengthening lashes, and/or increasing the diameter of lashes. Biotinoyl tripeptide-1 can also help shape lashes and helps provide the appearance of a curl to the lashes.

Various other components can also be used in the composition, each of which may or may not be considered an active ingredient. Additional components that can be used include one or more of the following: solvents, film forming polymers or copolymers, biotin, moisturizers and/or humectants, plankton extract, plant-based seed extracts, plant-based oils, and other additives.

In some embodiments, the composition comprises from about 20% to about 45%, or from about 20% to about 30%, by weight of a solvent. An exemplary solvent for use in the composition includes, but is not limited to, water. As can be appreciated, a solvent can be used to dissolve or dilute the other components of the composition. The solvent can also be used to help mix the remaining components and/or distribute them evenly throughout the composition. The solvent can also be considered a carrier for the remaining components of the composition.

In some embodiments, the composition comprises from about 10% to about 15% by weight of film forming polymers or copolymers. An exemplary film forming polymer or copolymer for use in the composition includes, but is not limited to, acrylates/ethylhexyl acrylate copolymer (which is a copolymer of ethylhexyl acrylate and one or more monomers of acrylic acid, methacrylic acid, or one of their simple esters). In some embodiments, the composition comprises from about 10% to about 15% by weight of the film forming polymer or copolymer. In certain of such embodiments, the composition comprises a mixture comprising a film forming polymer or copolymer. For instance, the composition can comprise a mixture comprising from about 10% to about 15% acrylates/ethylhexyl acrylate copolymer, about 10% to about 15% water, and about 0.1% to about 0.5% Laureth-21 by weight of the composition. The film forming polymer or copolymer can form a barrier on the lashes and can provide water resistance or waterproof qualities to the composition.

In some embodiments, the composition comprises from about 0.1% to about 1%, or from about 0.1% to about 0.5%, by weight of biotin. Biotin, also known as vitamin B7, is involved in a wide range of metabolic processes. Biotin can help regulate the body's keratin infrastructure, a protein needed for healthy lashes. When used in the compositions disclosed herein, biotin can help strengthen lashes to reduce breakage and promote lash growth.

The composition disclosed herein can further comprise one or more moisturizers and/or humectants. In one embodiment, the composition comprises a mixture of two or more moisturizers, such as a mixture of two or more of panthenol, shea butter (butyrospermum parkii), sodium hyaluronate, and glycerin.

In some of such embodiments, the composition comprises from about 1% and about 5%, or from about 1% and about 3%, by weight of panthenol. Panthenol is a derivative of pantothenic acid, which is also known as vitamin B5. Panthenol can exhibit moisturizing, soothing, and anti-inflammatory properties. When used in the compositions disclosed herein, panthenol can aid in hydrating and/or moisturizing lashes, which can protect lashes from environmental damage.

In some of such embodiments, the composition comprises from about 1% and about 5%, or from about 1% to about 3%, by weight of shea butter. Shea butter is rich in vitamin A and vitamin E, which can contributed to healthy lashes. Shea butter can exhibit moisturizing properties. When used in the compositions disclosed herein, shea butter can also aid in hydrating and/or moisturizing lashes, which can protect lashes from environmental damage.

In some of such embodiments, the composition comprises from about 0.01% and about 0.1%, or from about 0.03% to about 0.05%, by weight of sodium hyaluronate. Sodium hyaluronate is hydrophilic and attracts water molecules. Sodium hyaluronate exhibits moisturizing properties. When used in the compositions disclosed herein, sodium hyaluronate can aid in hydrating and/or moisturizing lashes, which can increase lash density and strength.

In some of such embodiments, the composition comprises from about 3% and about 5% by weight of glycerin. Glycerin attracts moisture can also aid in hydrating and/or moisturizing lashes, which can increase lash density and strength.

The composition disclosed herein can further comprise plankton extract, which is an extract of marine biomass that includes one or more of thalassoplankton, green micro-algae, greenish-blue algae, diatoms, and nitrogen-fixing seaweed. In some embodiments, the composition comprises from about 0.001% to about 0.01%, or from about 0.005% to about 0.01%, by weight of plankton extract. Plankton extract exhibits moisturizing and soothing properties. When used in the compositions disclosed herein, plankton extract can aid in nourishing and strengthening hair follicles, making them shinier and stronger. Plankton extract can also reduce lash fall out/loss by promoting increased density and volume. In certain embodiments, the composition comprises Symlash®1631 which comprises a mixture of about 0.1% to about 0.5% pentylene glycol and about 0.005% to about 0.01% plankton extract by weight of the composition.

The composition disclosed herein can further comprise one or more plant-based seed extracts. For example, in one embodiment, the composition comprises a mixture of two or more plant-based seed extracts, including Linum usitatissimum seed extract (flaxseed), and Salvia hispanica seed extract (chia seed).

In some embodiments, the composition comprises from about 1% to about 5%, or from about 1% to about 3%, by weight of flaxseed extract; and from about 0.1% to about 1%, or from about 0.5% to about 1%, by weight of chia seed extract. Plant-based seed extracts include fatty acids that can soften, smooth, and/or hydrate lashes.

The composition disclosed herein can further comprise one or more plant-based oils. For example, in one embodiment, the composition comprises a mixture of two or more plant-based oils, including lupines albus seed oil and Triticum vulgare germ oil. In some embodiments, the composition comprises from about 0.01% to about 0.1%, or from about 0.05% to about 0.1%, by weight of lupines albus seed oil; and from about 0.01% to about 0.1%, or from about 0.03% to about 0.05% by weight of Triticum vulgare germ oil. Plant-based oils such as lupines albus seed oil and Triticum vulgare germ oil are non-fragrant plant oils that can soften, smooth, and/or hydrate lashes.

Various other additives can be added as desired. Exemplary additives include one or more of pigments, thickeners, emulsifying agents, emollients, binders, pH adjusters, preservatives, stabilizing agents, chelating agents, anti-inflammatory agents, or combinations thereof. It will also be appreciated that any of the components identified throughout this disclosure can provide one or more additional properties to the composition.

Exemplary pigments that can be used include, but are not limited to, iron oxides. In some embodiments, the composition comprises from about 5% to about 10% by weight of pigments (such as iron oxides).

Exemplary thickeners that can be used include, but are not limited to, waxes such as carnauba wax. In some embodiments, the composition comprises from about 3% to about 5% by weight of wax (such as carnauba wax).

Exemplary emulsifying agents that can be used include, but are not limited to, glyceryl stearate SE and stearic acid. In some embodiments, the composition comprises from about 3% to about 5% by weight of glyceryl stearate SE, and from about 3% to about 8% of stearic acid.

Exemplary emollients that can be used include, but are not limited to, palmitic acid. In some embodiments, the composition comprises from about 1% to about 3% by weight of an emollient (e.g., palmitic acid).

Exemplary binders that can be used include, but are not limited to, polyvinylpyrrolidone (PVP). In some embodiments, the composition comprises from about 1% to about 3% by weight of a binder (e.g., polyvinylpyrrolidone).

Exemplary pH adjusters that can be used include, but are not limited to, tromethamine. In some embodiments, the composition comprises from about 1% to about 3% by weight of a pH adjuster (e.g., tromethamine).

Exemplary preservatives that can be used include, but are not limited to, phenoxyethanol. In some embodiments, the composition comprises from about 0.5% to about 1% by weight of a preservatives (e.g., phenoxyethanol).

Exemplary stabilizing agents that can be used include, but are not limited to, xanthan gum. In some embodiments, the composition comprises from about 0.1% to about 0.5% by weight of a stabilizing agent (e.g., xanthan gum).

Exemplary chelating agents that can be used include, but are not limited to, disodium ethylenediaminetetraacetic acid (disodium EDTA). In some embodiments, the composition comprises from about 0.1% to about 0.5% by weight of a chelating agent (e.g., disodium EDTA).

Exemplary anti-inflammatory agents that can be used include, but are not limited to, saw palmetto. In some embodiments, the composition comprises from about 0.005% to about 0.01% by weight of an anti-inflammatory agent (e.g., saw palmetto).

Methods of using the compositions are also disclosed herein. In particular, it is contemplated that any of the components, principles, and/or embodiments discussed above may be utilized in either a cosmetic composition or a method of using the same. An illustrative method of using a cosmetic composition, according to one embodiment of the present disclosure, includes obtaining a composition disclosed herein and applying one or more layers of the cosmetic composition to one's eyelashes using a brush or similar tool. At the desired time, the method can further comprise removing the composition via rinsing the lashes with water (e.g., warm water) and a washcloth or other material. Makeup remover can also optionally be used. Other methods and/or method steps are also contemplated.

Examples: The following examples are exemplary and are not intended to be limiting to or exhaustive of the embodiments disclosed herein.

### Example 1

A cosmetic composition was prepared comprising the following components:

| | % weight of | |
|---|---|---|
| Component | Composition | |
| SYMPEPTIDE ® Xlash | | |
| Water | 3-5 | % |
| Glycerin | 0.5-1 | % |
| Myristoyl Pentapeptide-17 | 0.005-0.01 | % |
| Benzoic Acid | 0.005-0.01 | % |
| Sodium Benzoate | 0.005-0.01 | % |
| WIDELASH ™ | | |
| Gylcerin | 1-3 | % |
| Water | 0.1-0.5 | % |
| Panthenol | 0.01-0.03 | % |
| Biotinoyl Tripeptide-1 | 0.005-0.01 | % |
| Water | 20-30 | % |
| Film Forming Polymer or Copolymer | | |
| Acrylates/Ethylhexyl Acrylate | 10-15 | % |
| Copolymer | | |
| Water | 10-15 | % |
| Laureth-21 | 0.1-0.5 | % |
| Biotin | 0.1-0.5 | % |
| Panthenol | 1-3 | % |
| Shea Butter | 1-3 | % |
| Sodium Hyaluronate | 0.03-0.05 | % |
| Glycerin | 3-5 | % |
| Symlash ® | | |
| Pentylene Gylcol | 0.1-0.5 | % |
| Plankton Extract | 0.005-0.01 | % |
| Plant-Based Seed Extracts | | |
| Flaxseed Extract | 1-3 | % |
| Chia Seed Extract | 0.5-1 | % |
| Plant-Based Oils | | |
| Lupines Albus Seed Oil | 0.05-0.1 | % |
| Triticum Vulgare Germ Oil | 0.03-0.05 | % |
| Pigments | 5-10 | % |
| Carnauba Wax | 3-5 | % |
| Glyceryl Stearate SE | 3-5 | % |
| Stearic Acid | 3-8 | % |
| Palmitic Acid | 1-3 | % |
| Polyvinylpyrrolidone | 1-3 | % |
| Tromethamine | 1-3 | % |
| Phenoxyethanol | 0.5-1 | % |
| Xanthan Gum | 0.1-0.5 | % |
| Disodium EDTA | 0.1-0.5 | % |
| Saw Palmetto | 0.005-0.01 | % |
| VP/Eicosene Copolymer | 3-5 | % |
| Star-Shape Fiber | | |
| Nylon 6 | 0.1-0.5 | % |
| Silica | 0.001-0.003 | % |
| Iron Oxides | 0.001-0.003 | % |

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. Further, all ranges include both endpoints.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. The scope of the invention is therefore defined by the following claims and their equivalents.

1. A cosmetic composition, comprising:
myristoyl pentapeptide-1;
biotinoyl tripeptide-1;
one or more solvents;
one or more film forming polymers;
biotin;
one or more moisturizes and/or humectants;
plankton extract;
one or more plant-based seed extracts; and
one or more plant-based oils.

2. The composition of claim 1, wherein the composition comprises SYMPEPTIDE® Xlash comprising the myristoyl pentapeptide-17.

3. The composition of claim 1, wherein the composition comprises WIDELASH™ comprising the biotinoyl tripeptide-1.

4. The composition of claim 1, wherein the one or more solvents comprises water.

5. The composition of claim 1, wherein the one or more film forming polymers comprises acrylates/ethylhexyl acrylate copolymer.

6. The composition of claim 1, wherein the one or more moisturizers and/or humectants comprises a mixture of two or more of panthenol, shea butter, sodium hyaluronate, and glycerin.

7. The composition of claim 1, wherein the one or more plant-based seed extracts comprise a mixture of flaxseed extract and chia seed extract.

8. The composition of claim 1, wherein the one or more plant-based oils comprise a mixture of lupines albus seed oil and Triticum vulgare germ oil.

9. The composition of claim 1, further comprising a tubing fiber or polymer comprising VP/Eicosene.

10. The composition of claim 1, further comprising a star-shaped fiber or polymer comprising nylon-6.

11. The composition of claim 1, configured for use as a lash serum, primer, and mascara all in one.

12. The composition of claim 1, wherein the composition is free of prostaglandins.

13. A cosmetic composition for eyelashes, comprising:
myristoyl pentapeptide-1;
biotinoyl tripeptide-1;
one or more solvents;
one or more film forming polymers; and
one or more tubing fiber or polymer.

14. The composition of claim 13, further comprising one or more star-shaped fiber or polymer.

15. The composition of claim 13, wherein the composition comprises SYMPEPTIDE® Xlash comprising the myristoyl pentapeptide-17.

16. The composition of claim 13, wherein the composition comprises WIDELASH™ comprising the biotinoyl tripeptide-1.

17. The composition of claim 13, configured for use as a lash serum, primer, and mascara all in one.

18. The composition of claim 13, wherein the cosmetic composition is free of prostaglandins.

19. A method of applying a cosmetic, comprising:
obtaining a cosmetic composition comprising:
myristoyl pentapeptide-1;
biotinoyl tripeptide-1;
one or more solvents;
one or more film forming polymers; and
one or more tubing fiber or polymer; and
applying the cosmetic composition to an eyelash.

20. The method of claim 19, wherein the cosmetic composition is free of prostaglandins.
